(19) United States
(12) Patent Application Publication
(10) Pub. No.: US 2013/0217655 A1
(43) Pub. Date: Aug. 22, 2013

(54) COMPOSITIONS FOR TREATING PSORIASIS OF THE SCALP

(71) Applicant: Zeev Even-Chen, Rehovot (IL)

(72) Inventor: Zeev Even-Chen, Rehovot (IL)

(73) Assignee: Dermipsor Ltd., Katzrin (IL)

(21) Appl. No.: 13/878,669

(22) PCT Filed: Oct. 10, 2011
(86) PCT No.: PCT/IL11/00797

§ 371 Date: May 9, 2013

Related U.S. Application Data
(60) Provisional application No. 61/391,641, filed on Oct. 10, 2010.

(51) Int. Cl. A61K 31/593; A61K 31/455

(52) U.S. Cl. CPC A61K 31/593; A61K 31/455
USPC 514/168

(57) ABSTRACT

Compositions useful for the treatment of hyperproliferative dermal diseases of the scalp. Specifically, topical gel and liquid compositions that include calcipotriol and nicotinamide in a polyethylene glycol-based carrier, for topical application to the scalp.

### FIELD OF THE INVENTION

The present invention relates to compositions useful for the treatment of hyperproliferative dermal diseases of the scalp. Specifically, the present invention provides liquid and gel compositions comprising calcipotriol and nicotinamide in a PEG-based carrier, for topical application to the scalp.

### BACKGROUND OF THE INVENTION

Psoriasis is a non-contagious chronic disorder which affects the skin and joints. Up to 2% of the world's population suffers from psoriasis. It is estimated that 100 million people worldwide suffer from psoriasis, with 23 million psoriasis patients in U.S. and Europe alone,

Psoriasis commonly causes red scaly patches to appear on the skin. The scaly patches caused by psoriasis, called psoriatic plaques, are areas of inflammation and excessive skin production. Skin rapidly accumulates at these sites and takes a silvery-white appearance. Plaques frequently occur on the skin of the elbows and knees, but can affect any area including the scalp and genitals. Psoriasis is hypothesized to be immune-mediated and is not contagious.

The disorder is a chronic recurring condition which varies in severity from minor localized patches to complete body coverage. About 85% of patients with plaque psoriasis have Mild to Moderate forms of the disease. Fingernails and toenails are frequently affected (psoriatic nail dystrophy)—and can be seen as an isolated finding. Psoriasis can also cause inflammation of the joints, which is known as psoriatic arthritis. Ten to fifteen percent of people with psoriasis have psoriatic arthritis.

Scalp psoriasis is very common. In fact at least half of the people who have psoriasis have it on their scalp. As with psoriasis elsewhere on the body, skin cells grow too quickly on the scalp and cause red lesions covered with scale to appear.

Scalp psoriasis can be very mild, with slight fine scaling. It can also be very severe with thick crusted plaques covering the entire scalp, which commonly can cause hair loss. Psoriasis can extend beyond the hairline onto the forehead, the back of the neck and around the ears (a common area). Most of the time, people with scalp psoriasis have psoriasis on other parts of their body as well. But for some, the scalp is the only affected area.

Psoriasis is caused by unknown factors that stimulate T-lymphocyte activation, proliferation, and cytokine release that leads to hyperproliferation of keratinocytes. Although the etiology of psoriasis is unknown, it is believed to have a genetic component. The affected keratinocytes are responsible for the typical clinical features of the disease: well-demarcated inflamed skin lesions covered with a silvery white scale, covering variable areas of the body surface. Psoriasis primarily affects adults and may manifest itself in different variations and degrees of severity.

Several factors are thought to aggravate psoriasis. These include stress, excessive alcohol consumption, and smoking. Individuals with psoriasis may suffer from depression and loss of self-esteem. As such, quality of life is an important factor in evaluating the severity of the disease. There are many treatments available but because of its chronic recurrent nature psoriasis is a challenge to treat.

The main strategy for the treatment of hyperproliferative skin diseases, including psoriasis, is to prevent excessive keratinocyte division and to stimulate cell differentiation.

At present there are three modalities of treatment for psoriasis:

1) Topical administration of therapeutic cream or ointment, for treating mild and moderate cases. Such formulations typically incorporate active ingredients such as steroids, vitamin D analogs, coal tar based compounds or anthralin.

2) Phototherapy (natural or artificial UVA or UVB) used alone or in combination with systemic medication or topical treatment, requires repeated visits to the clinic and exposes the patient to the concomitant dangers of radiation.

3) Systemic treatment, relying on immunosuppressive therapy, is limited to severe cases due to the serious side effects. These medications include retinoids such as Soriatane® and Accutane®; TNFα blockers, such as Enbrel® and Remicade; the antibody based T-cell modulator Efalizumab®, and the immunosuppressant drug methotrexate (Trexall®).

Topical treatments are the mainstay approach for of the treatment of scalp psoriasis, with the vehicle as well as the active ingredient being relevant for efficacy, tolerability and compliance. Vehicles can be shampoos, lotions, gels, foams, creams and more greasy ointments. Active ingredients contained in prior art treatments for scalp psoriasis include salicylic acid, coal tar (liquor carbonis detergents), dithranol, corticosteroids, imidazole derivatives, retinoic acid derivatives, vitamin D₃ analogs and combination of vitamin D₃ analogs with corticosteroids. Various combination treatments are also known, such as coal tar with salicylic acid, betamethasone with salicylic acid and vitamin D₃ analogs with betamethasone dipropionate. Many of these medications, such as coal tar and corticosteroids, can have serious side effects, particularly with long term use. Others, such as salicylic acid merely treat a symptom of the disease i.e. scaling, and have no effect on the underlying cause of the disease.

### Vitamin D₃ Analogs and Derivatives

Vitamin D is a prohormone with several active metabolites that act as hormones. In the skin, previtamin D₃ is synthesized photochemically from 7-dehydrocholesterol and is slowly isomerized to vitamin D₃, which is removed by vitamin D-binding protein. In the liver, vitamin D₃ is converted to 25(OH)D₃ (25-hydroxycholecalciferol), the major circulating form, which passes through the enterohepatic circulation and is reabsorbed from the gut. In the kidneys, it is further hydroxylated to the more metabolically active form, 1α,25(OH)₂D₃ (1α,25-dihydroxycholecalciferol, calcitriol, vitamin D hormone).

Experimental evidence has shown that vitamin D functions as an anti-proliferative agent and stimulates the terminal differentiation of keratinocytes. In psoriatic lesions, epidermal keratinocytes exhibit hyperproliferation and impaired differentiation triggered by inflammation. Therefore, vitamin D and certain analogs are effective in treating psoriasis vulgaris (reviewed in DeLuca 1988; Lehmann et al., 2004). The systemic administration of these compounds is limited by their toxicity and adverse effect on calcium metabolism, therefore topical preparations are preferred.

Calcipotriol (also known as calcipotriene), is a vitamin D₃ analog which is marketed in the United States under the trade name Dovonex®, and in Europe under the trade name Daivonex®, as a topical antipsoriatic preparation. Calcipotriol is as potent as the naturally occurring calcitriol in regulating cell proliferation, but has the benefit of being much less active in its effect on calcium metabolism. Despite this, calcipotriol on its own is only partially effective in treating psoriatic lesions. Accordingly, calcipotriol is used in combination with steroid for more efficient treatment of psoriasis, for example in the product Taclonex® (calcipotriene and betamethasone dipropionate). Such a product however, is disadvantageous and not suitable for long term use due to the undesirable side effects of the steroid.

The art provides some examples of vitamin D analogs and derivatives and compositions comprising the same.

Vitamin D analogs are described in U.S. Pat. No. 4,851,401 (cyclopentano-vitamin D analogs), U.S. Pat. No. 5,120,722 (trihydroxycalciferol derivatives), U.S. Pat. No. 5,446,035 (20-methyl substituted vitamin D), U.S. Pat. No. 5,411,949 (23-oxa-derivatives), U.S. Pat. No. 5,237,110 (19-nor-vitamin D compounds), U.S. Pat. No. 4,857,518 (hydroxylated 24-homo-vitamin D derivatives). Additional Vitamin D analogs are taught in U.S. Pat. Nos. 4,804,502; 4,866,048; 5,145,846 5,374,629; 5,403,940; 5,446,034; and 5,447,924.

U.S. Pat. No. 5,037,816 is directed to a method of treating psoriasis which comprises topically administering an effective amount of a vitamin D compound which is capable of stimulating the differentiation of cultured tumor cells or normal rodent or human fibroblasts or keratinocytes in vitro.

U.S. Pat. No. 6,552,009 discloses a composition comprising a vitamin D analog and a derivative of retinoid useful in treating disorders characterized by abnormal cell proliferation and/or cell differentiation. In certain preferred embodiments the vitamin D analog is selected from calcitriol and calcipotriol.

U.S. Pat. No. 6,753,013 describes a pharmaceutical composition for dermal use comprising a combination of a vitamin D analog and a corticosteroid, the composition alleviating the inconvenience of a two-component regimen for the treatment of psoriasis and other inflammatory skin diseases.

U.S. Pat. No. 6,787,529 discloses a non-aqueous pharmaceutical gel composition for application on skin, said composition comprising at least one vitamin D analog inter alia calcipotriol, a corticosteroid, a viscosity increasing excipient and at least one solvent inter alia a propylene glycol diester, and use of the composition for topical treatment of psoriasis and related conditions of the scalp in humans.

U.S. Pat. No. 5,834,016 discloses a liposome-based formulation comprising: vitamin D or a derivative thereof selected from 1α,25-dihydroxycholecalciferol, 1α-hydroxycholecalciferol and mixtures thereof; lecithin or hydrogenated lecithin, and cholesterol or a derivative thereof inter alia polyethylene glycol derivatives of cholesterol, for use as a pharmaceutical preparation for the treatment of psoriasis.

U.S. Pat. No. 5,789,399 discloses a method for treating pruritus, for example, that caused by psoriasis, comprising topically administering a formulation which is an emulsion comprising water, a water-insoluble organic liquid, a surface active agent inter alia a PEG-ester, and a vitamin D compound or analog thereof.

### Nicotinamide

Nicotinamide (NA, niacinamide), a derivative of vitamin B₃ and a precursor of the coenzyme nicotinamide adenine dinucleotide (NAD), displays multiple functions in cell metabolism. NA has been shown to induce the differentiation of insulin-producing cells (Otonkoski et al, 1993) and the protection of the pancreatic beta-cells from genotoxic agents (Pipeleers and Van de Winkel, 1986).

U.S. Pat. No. 5,914,334 discloses topical gel formulations for treatment of acne and psoriasis, the formulations being free of propylene glycol, and comprising ethyl-6-[2-(4,4-dimethylthiochroman-6-yl)-ethynyl]nicotinate in a specific carrier, the carrier comprising inter alia poloxamer 407, polyethylene glycol, polysorbate 40 and hexylene glycol.

U.S. Pat. No. 6,248,763 relates to specific topical compositions for treating skin conditions for example acne and psoriasis, which comprise 0.01%4% methyl nicotinate, as the active ingredient.

U.S. Pat. No. 4,067,975 discloses a method for alleviating the symptoms of psoriasis in a human, comprising topically applying a composition comprising a 6-substituted nicotinamide inter alia 6-aminonicotinamide, or thionicotinamide, at a concentration of 0.01 to 5% in a pharmaceutically acceptable carrier. According to the disclosure, the carrier may comprise inter alia a mixture of a liquid and an ointment base or a mixture of a liquid and a water-in-oil lotion.

### Combinations of Nicotinamide and Vitamin D₃ Analogs

The synergistic effects of NA and vitamin D₃ analogs on differentiation and proliferation of human epidermal cells have been reported by some of the present inventors. International Patent Publication No. WO 01/51051 is directed to compositions and methods for treating a hyperproliferative epidermal pathology inter alia psoriasis, using as an active agent a combination of nicotinamide and a metabolite of vitamin D₃, such as 1α25(OH₂)D₃.

International Patent Publication No. WO 2004/006887 discloses a method of treating a benign or malignant hyperproliferative epidermal pathology, comprising administration of a therapeutically effective amount of nicotinamide, or an agonist, derivative, metabolite or prodrug thereof, in combination with vitamin D₃, or an agonist, derivative, metabolite or prodrug thereof. According to the disclosure, a benignhyperproliferative epidermal pathology is inter alia psoriasis.

International Patent Publication No. WO 2006/120681 discloses topical dermatological compositions for preventing, retarding, arresting or reversing atrophy in mammalian skin, and for preventing or treating an epidermal condition related to aging. The disclosed compositions comprise a first agent selected from vitamin D₃ and a vitamin D₃ analog, and a second agent which may be nicotinamide, or an agonist, derivative, metabolite or prodrug thereof. According to the disclosure, the vitamin D₃ analog may be calcipotriol.

International Patent Publication No. WO 2006/120682 discloses topical pharmaceutical compositions useful in treating hyperproliferative skin diseases, inter alia psoriasis, the compositions consisting essentially of specific combinations of calcipotriol, nicotinamide, and a polyethylene glycol-based carrier. According to the disclosure, the compositions comprise about 10 μg/g to about 100 μg/g calcipotriol; about 0.5 to about 25 mg/g nicotinamide; about 70% to about 80% (w/w) polyethylene glycol (PEG)-400, and about 15% to about 25% (w/w) PEG-4000. However, this publication teaches away from the present invention, since it discloses that combinations of calcipotriol and nicotinamide are most effective when the concentration of nicotinamide is below 10 mg/g. Furthermore, this publication does not address use of the disclosed formulations for use in the treatment of scalp psoriasis.

Vitamin D₃ and vitamin D₃ analogs are known to possess anti-proliferative and prodifferentiating properties and are therefore effective in the treatment of hyperproliferative skin disorders, for example psoriasis vulgaris. The synergistic effect of vitamin D₃ with nicotinamide to further inhibit proliferation of keratinocytes has been demonstrated by some of the inventors of the present invention. Although specific formulations comprising vitamin D₃ derivatives and nicotinamide are known, optimization of the two components in a pharmaceutical composition for the treatment of hyperproliferative disorders of the scalp has never been shown.

The art has neither taught nor suggested the specific formulations disclosed herein for the prevention, treatment or attenuation of disorders associated with hyperproliferative disorders of the scalp.

There remains a yet unmet medical need for compositions and methods useful in preventing and treating the symptoms associated with scalp psoriasis.

### SUMMARY OF THE INVENTION

The present invention provides liquid and gel compositions comprising calcipotriol and nicotinamide in a specific PEG-based carrier, and methods of use thereof for the treatment of hyperproliferative diseases of the scalp, in particular psoriasis.

Calcipotriol, when provided in combination with nicotinamide, produces a synergistic effect in reducing the symptoms of hyperproliferative disease in a specific concentration range. Accordingly, the present invention provides highly efficacious therapeutic compositions consisting essentially of calcipotriol and nicotinamide, at defined concentration ranges.

The inventor of the present invention has previously disclosed PEG-based ointment formulations comprising as active ingredients calcipotriol and nicotinamide. However, the inventor has unexpectedly found that by use of a formulation comprising nicotinamide at a concentration greater than 10 mg/g, and by utilizing a different PEG-based carrier, the compositions provide enhanced therapeutic efficacy for treatment of scalp psoriasis, as compared to the aforementioned prior art formulations comprising the same active ingredients. More specifically, the compositions of the present invention provide a substantially higher dose of nicotinamide, and are formulated in a carrier having a higher concentration of PEG-400, and a lower concentration of PEG-4000 (or completely lacking PEG-4000) in comparison to the preferred formulations of the prior art. The compositions of the present invention are further distinguished over those of the prior art by requiring a solvent such as propylene glycol. By manipulating the relative concentrations of the PEG-400 and PEG-4000, either liquid or gel formulations are obtained, which are advantageous for application to the scalp, as opposed to prior art ointment formulations which can be excessively sticky and hinder patient compliance. Furthermore, the compositions disclosed herein have been found to provide a superior treatment for scalp psoriasis, even in cases which are refractory to treatment using the prior art compositions of the inventors. In one aspect, the present invention provides a pharmaceutical composition for topical administration to the scalp, the composition comprising:

calcipotriol at a concentration of about 10 μg/g (micrograms per gram) to about 100 μg/g;

nicotinamide at a concentration of about 10 mg/g to about 25 mg/g; and

a dermatologically acceptable carrier, wherein the carrier comprises:

polyethylene glycol (PEG)-400 at a concentration of about 75% to about 95% (w/w);

an additional solvent at a concentration of about 2% to about 25% (w/w);

a surfactant at a concentration of about 1% to about 5% (w/w); and optionally,

polyethylene glycol (PEG)-4000 at a concentration of about 0.1% to about 12% (w/w). In one embodiment, calcipotriol is provided at a concentration of about 20 μg/g to about 80 μg/g. In one embodiment, calcipotriol is provided at a concentration of about 30 μg/g to about 60 μg/g. In a currently preferred embodiment, the calcipotriol is provided at a concentration of about 50 μg/g.

In a preferred embodiment, nicotinamide is provided at a concentration of about 10 mg/g to about 20 mg/g. In a more preferred embodiment, nicotinamide is provided at a concentration of about 10 mg/g to about 18 mg/g. In other preferred embodiments, nicotinamide is provided at a concentration of about 12 mg/g to about 16 mg/g, or about 13 mg/g to about 15 mg/g. In one currently preferred embodiment, nicotinamide is provided at a concentration of about 14 mg/g.

In a currently preferred embodiment, calcipotriol is provided at a concentration of about 50 μg/g, and nicotinamide is provided at a concentration of about 14 mg/g.

In one embodiment, PEG-400 is provided at a concentration of about 78% to about 93% (w/w). In particular embodiments, PEG-400 is provided at a concentration of about 88%, or about 90%, or about 93%.

In one embodiment, PEG-4000 is provided at a concentration of about 1% to about 12% (w/w) and the composition is in the form of a gel. In particular embodiments, PEG-4000 is provided at a concentration of about 4%, or about 6%, or about 8%. In another embodiment, PEG-4000 is provided at a concentration of about 0.1% to about 0.5% (w/w) and the composition is in the form of a liquid. In an alternate embodiment, the composition is substantially devoid of PEG-4000 and the composition is in the form of a liquid.

In one embodiment, PEG-400 is provided at a concentration of about 78% to about 93% (w/w) and PEG-4000 is provided at a concentration of about 1% to about 12% (w/w). In another embodiment, PEG-400 is provided at a concentration of about 78% to about 93% (w/w) and PEG-4000 is provided at a concentration of about 0.1% to about 0.5% (w/w).

In one embodiment, the solvent is selected from the group consisting of: propylene glycol, polypropylene glycol, glycerin, sorbitol esters, 1,2,6-hexanetriol, ethanol, isopropanol, diethyl tartrate, butanediol, water and mixtures thereof. In one embodiment, the solvent is propylene glycol.

In one embodiment, the propylene glycol is provided at a concentration of about 2% to about 25% (w/w). In particular embodiments, propylene glycol is provided at a concentration of about 5%, or about 10%, or about 20%.

In one embodiment, the surfactant is selected from the group consisting of: a polyethoxylated alcohol e.g. Steareth; a fatty alcohol e.g. cetyl/stearyl alcohol; a polyoxyethylene glycol ester; a polyoxethylene sorbitan fatty acid ester e.g. polysorbate-60; hydrogenated castor oil; cetyl trimethyl ammonium bromide; cetyl trimethyl ammonium chloride; an alkyl benzene sulphonate, sodium dodecyl sulfate; sodium sulfosuccinate; sodium lauryl sulfate; an alkyl naphthalene sulfonate condensate sodium salt; sodium stearate; egg lecithin; soya bean lecithin; a synthetic saturated lecithin e.g. dimyristoyl phosphatidyl choline; a synthetic unsaturated lecithin e.g. dioleyl phosphatidyl choline; an ethoxylated sorbitan ester; a sorbitan ester; a polyglycerol ester; a sucrose ester; a poloxamer; an alkyl polyglucoside; a polyalkyleneoxide modified heptamethyltrisiloxane; an allyloxypolyethylene glycol methylether and mixtures thereof. In one embodiment, the surfactant is polyoxyethylated stearyl alcohol (Steareth-20). In one embodiment, the Steareth-20 is provided at a concentration of about 2% (w/w).

In a preferred embodiment, the carrier comprises PEG-400, PEG-4000, propylene glycol and Steareth-20. In one embodiment, the composition of the present invention comprises calcipotriol, nicotinamide, PEG-400, PEG-4000, propylene glycol and Steareth-20.

In one embodiment, the composition is in the form of a gel. In particular embodiments the gel is selected from the group consisting of a hydrogel, a macrogel, a microgel, a nanogel, an emulsion, a microemulsion, a suspension, a spray, and combinations thereof. In one embodiment, the composition in the form of a gel comprises PEG-400 at a concentration of about 78% to about 93% (w/w), PEG-4000 at a concentration of about 1% to about 12% (w/w) and propylene glycol at a concentration of about 5% (w/w).

In one embodiment, the composition is in the form of a liquid. In one embodiment, the liquid is selected from the group consisting of an aqueous solution, a non-aqueous solution, a lotion, a spray, an emulsion, and a microemulsion. In one embodiment, the composition in the form of a liquid comprises PEG-400 at a concentration of about 78% to about 93% (w/w), PEG-4000 at a concentration of about 0.1% to about 0.5% (w/w) and propylene glycol at a concentration of about 5% (w/w). In another embodiment, the composition in the form of a liquid comprises PEG-400 at a concentration of about 78% to about 93% (w/w), propylene glycol at a concentration of about 5% (w/w) to about 20% (w/w) and is substantially devoid of PEG-4000.

In one embodiment, the gel composition has the formulation:
about 10 μg/g to about 100 μg/g calcipotriol;
about 10 to about 18 mg/g nicotinamide;
about 75% to about 95% (w/w) PEG-400;
about 1% to about 12% (w/w) PEG-4000;
about 2% to about 25% (w/w) propylene glycol;
about 1% to about 5% (w/w) Steareth-20; and
about 0.1 to about 1% (w/w) vitamin E.

In one embodiment, the gel composition has the formulation:
about 50 μg/g calcipotriol;
about 14 mg/g nicotinamide;
about 75% to about 95% (w/w) PEG-400;
about 1% to about 12% (w/w) PEG-4000;
about 2% to about 25% (w/w) propylene glycol;
about 1% to about 5% (w/w) Steareth-20; and
about 0.1 to about 1% (w/w) vitamin E.

In a particular embodiment, the gel composition has the formulation:
calcipotriol at a concentration of about 50 μg/g;
nicotinamide at a concentration of about 14 mg/g;
PEG-400 at a concentration of about 88.7%;
PEG-4000 at a concentration of about 4.0%;
propylene glycol at a concentration of about 5%;
Steareth-20 at a concentration of about 2%; and
vitamin E at a concentration of about 0.3%.

In a particular embodiment, the gel composition has the formulation:
calcipotriol at a concentration of about 50 μg/g;
nicotinamide at a concentration of about 14 mg/g;
PEG-400 at a concentration of about 89.7%;
PEG-4000 at a concentration of about 3.0%;
propylene glycol at a concentration of about 5%;
Steareth-20 at a concentration of about 2%; and
vitamin E at a concentration of about 0.3%.

In a particular embodiment, the gel composition has the formulation:
calcipotriol at a concentration of about 50 μg/g;
nicotinamide at a concentration of about 14 mg/g;
PEG-400 at a concentration of about 91.7%;
PEG-4000 at a concentration of about 1.0%;
propylene glycol at a concentration of about 5%;
Steareth-20 at a concentration of about 2%; and
vitamin E at a concentration of about 0.3%.

In one embodiment, the liquid composition has the formulation:
about 50 μg/g calcipotriol;
about 14 mg/g nicotinamide;
about 75% to about 95% (w/w) PEG-400;
about 2% to about 25% (w/w) propylene glycol;
about 1% to about 5% (w/w) Steareth-20; and
about 0.1 to about 1% (w/w) vitamin E; and optionally
about 0.1% to about 0.5% (w/w) PEG-4000.

In a particular embodiment, the liquid composition has the formulation:

calcipotriol at a concentration of about 50 μg/g; nicotinamide at a concentration of about 14 mg/g;
PEG-400 at a concentration of about 92.7%;
propylene glycol at a concentration of about 5%;
Steareth-20 at a concentration of about 2%; and
vitamin E at a concentration of about 0.3%.

In a particular embodiment, the liquid composition has the formulation:
calcipotriol at a concentration of about 50 μg/g;
nicotinamide at a concentration of about 14 mg/g;
PEG-400 at a concentration of about 87.7%;
propylene glycol at a concentration of about 10%;
Steareth-20 at a concentration of about 2%; and
vitamin E at a concentration of about 0.3%.

In a particular embodiment, the liquid composition has the formulation:
calcipotriol at a concentration of about 50 μg/g;
nicotinamide at a concentration of about 14 mg/g;
PEG-400 at a concentration of about 77.7%;
propylene glycol at a concentration of about 20%;
Steareth-20 at a concentration of about 2%; and
vitamin E at a concentration of about 0.3%.

In a particular embodiment, the liquid composition has the formulation:
calcipotriol at a concentration of about 50 μg/g;
nicotinamide at a concentration of about 14 mg/g;
PEG-400 at a concentration of about 92.5%;
PEG-4000 at a concentration of about 0.2%;
propylene glycol at a concentration of about 5%;
Steareth-20 at a concentration of about 2%; and
vitamin E at a concentration of about 0.3%.

In a particular embodiment, a kit comprises the liquid or gel composition of the invention packaged in a container suitable for dispensing of said composition; and written instructions for use. In a particular embodiment, the liquid or gel composition of the invention is formulated as a spray. In a particular embodiment, the spray is provided in a spray dispenser.

In another aspect, the present invention provides a method of preventing or treating a hyperproliferative skin disease or disorder of the scalp, the method comprising the step of:

topically administering to the scalp of a subject in need thereof a therapeutically effective amount of a composition, the composition comprising calcipotriol at a concentration of about 10 μg/g to about 100 μg/g; nicotinamide at a concentration of about 10 mg/g to about 25 mg/g; and a dermatologically acceptable carrier, wherein the carrier comprises PEG-400 at a concentration of about 75% to about 95% (w/w); propylene glycol at a concentration of about 2% to about 25% (w/w), and a surfactant at a concentration of about 1% to about 5%; and optionally, PEG-4000 at a concentration of about 0.1% to about 12% (w/w);

thereby preventing or treating the hyperproliferative skin disease or disorder of the scalp.

In a particular embodiment, the method comprises topical administration to the scalp up to about four times daily of a therapeutically effective amount of said composition. In a particular embodiment, the composition is administered once or twice daily. In one embodiment, the composition is administered twice daily at intervals of about 12 hours. In another embodiment, the composition is administered once daily. In another embodiment, the composition is administered three to five times per week.

In a particular embodiment, the disease or disorder of the scalp is selected from the group consisting of: psoriasis, solar (actinic) keratosis, naevus sebaceous, seborrhoeic keratoses, atopic dermatitis and lichen planus.

In a particular embodiment, the calcipotriol is provided at a concentration of about 20 μg/g to about 80 μg/g. In another embodiment, calcipotriol is provided at a concentration of about 30 μg/g to about 60 μg/g. In a particular embodiment, the calcipotriol is provided at a concentration of about 50 μg/g.

In a particular embodiment, nicotinamide is provided at a concentration of about 10 mg/g to about 20 mg/g. In a particular embodiment, nicotinamide is provided at a concentration of about 10 mg/g to about 18 mg/g. In other particular embodiments, nicotinamide is provided at a concentration of about 12 mg/g to about 16 mg/g, or about 13 mg/g to about 15 mg/g. In a particular embodiment, nicotinamide is provided at a concentration of about 14 mg/g. In a particular embodiment, the calcipotriol is provided at a concentration of about 50 μg/g; and the nicotinamide is provided at a concentration of about 14 mg/g.

In some embodiments, PEG-400 is provided at a concentration of about 78% to about 93% (w/w). In particular embodiments, PEG-400 is provided at a concentration of about 88%, or about 90% or about 93%.

In one embodiment, PEG-4000 is provided at a concentration of about 1% to about 12% (w/w). In particular embodiments, PEG-4000 is provided at a concentration of about 4%, or about 6%, or about 8%. In another embodiment, PEG-4000 is provided at a concentration of about 0.1% to about 0.5% (w/w). In an alternate embodiment, the composition is substantially devoid of PEG-4000. In particular embodiments, propylene glycol is provided at a concentration of about 5%, or about 10%, or about 20%.

In one embodiment, the surfactant is polyoxyethylated stearyl alcohol (steareth). In particular embodiments, the composition is in a form selected from the group consisting of a gel and a liquid. In particular embodiments, the gel is selected from the group consisting of a hydrogel, a macrogel, a microgel, a nanogel, an emulsion, a microemulsion, a suspension, a spray, and combinations thereof. In one embodiment, the composition in the form of a gel comprises PEG-400 at a concentration of about 78% to about 93% (w/w), PEG-4000 at a concentration of about 1% to about 12% (w/w) and propylene glycol at a concentration of about 5% (w/w).

In one embodiment, the liquid composition is provided in a form selected from the group consisting of an aqueous solution, a non-aqueous solution, a lotion, a spray, an emulsion, and a microemulsion. In one embodiment, the composition in the form of a liquid comprises PEG-400 at a concentration of about 78% to about 93% (w/w), PEG-4000 at a concentration of about 0.1% to about 0.5% (w/w) and propylene glycol at a concentration of about 5% (w/w). In another embodiment, the composition in the form of a liquid comprises PEG-400 at a concentration of about 78% to about 93% (w/w), propylene glycol at a concentration of about 5% (w/w) to about 20% (w/w) and is substantially devoid of PEG-4000.

In one embodiment, the composition is a gel and has the formulation:
about 50 μg/g calcipotriol;
about 14 mg/g nicotinamide;
about 75% to about 95% (w/w) PEG-400;
about 1% to about 12% (w/w) PEG-4000;
about 2% to about 25% (w/w) propylene glycol;
about 1% to about 5% (w/w) Steareth-20; and
about 0.1 to about 1% (w/w) vitamin E.

In a particular embodiment, the gel composition has the formulation:
calcipotriol at a concentration of about 50 μg/g;
nicotinamide at a concentration of about 14 mg/g;
PEG-400 at a concentration of about 88.7%;
PEG-4000 at a concentration of about 4.0%;
propylene glycol at a concentration of about 5%;
Steareth-20 at a concentration of about 2%; and
vitamin E at a concentration of about 0.3%.

In a particular embodiment, the liquid composition has the formulation:
calcipotriol at a concentration of about 50 μg/g;
nicotinamide at a concentration of about 14 mg/g;
PEG-400 at a concentration of about 92.5%;
PEG-4000 at a concentration of about 0.2%;
propylene glycol at a concentration of about 5%;
Steareth-20 at a concentration of about 2%; and
vitamin E at a concentration of about 0.3%.

In a particular embodiment, the liquid composition has the formulation:
calcipotriol at a concentration of about 50 μg/g;
nicotinamide at a concentration of about 14 mg/g;
PEG-400 at a concentration of about 92.7%;
propylene glycol at a concentration of about 5%;
Steareth-20 at a concentration of about 2%; and
vitamin E at a concentration of about 0.3%.

In particular embodiments, the method is carried out for a period of about 4 weeks to about 52 weeks. In particular embodiments, the period is about 6 weeks to, about 12 weeks, or about 12 weeks to about 26 weeks.

In a particular embodiment, the subject is a mammal. In a particular embodiment, the subject is a human.

In another aspect, the invention provides the use of calcipotriol at a concentration of about 10 μg/g to about 100 μg/g; nicotinamide at a concentration of about 10 mg/g to about 25 mg/g; and a carrier, wherein the carrier comprises PEG-400 at a concentration of about 75% to about 95% (w/w); propylene glycol at a concentration of about 2% to about 25% (w/w), and a surfactant at a concentration of about 1% to about 5% (w/w), and optionally PEG-4000 at a concentration of about 0.1% to about 12% (w/w); for the preparation of a composition for the topical treatment of a hyperproliferative skin disease or disorder of the scalp.

In a particular embodiment, the calcipotriol is provided at a concentration of about 50 μg/g; and the nicotinamide is provided at a concentration of about 14 mg/g. Additional embodiments of the composition are as hereinbefore described.

Other objects, features and advantages of the present invention will become clear from the following description and drawings.

### DETAILED DESCRIPTION OF THE INVENTION

The present invention provides compositions comprising calcipotriol and nicotinamide as active ingredients, in a specific PEG-based carrier, and methods of use thereof for the treatment of hyperproliferative diseases of the scalp, in particular psoriasis.

Among psoriasis sufferers, a large proportion thereof have scalp involvement, which is particularly difficult to treat. The inventors of the present invention have previously disclosed topical compositions for treatment of psoriasis which comprise as active ingredients calcipotriol and nicotinamide. While the present inventors have observed that use of their prior art compositions for treatment of psoriasis at various body sites is highly effective, many cases of scalp psoriasis are refractory to such treatment, even in patients who demonstrate improvement in their psoriasis at other sites. The inventors of the present invention have now unexpectedly found that by adjusting the specific concentration of the active ingredient nicotinamide, and by using a different PEG-based carrier that provides liquid or gel formulations, the compositions of the present invention provide a higher degree of therapeutic efficacy for treatment of scalp psoriasis, as compared to prior art PEG-based ointment formulations comprising the same active ingredients.

Moreover, the compositions disclosed herein offer the following additional advantages over prior art compositions:

a) Dual anti-proliferative and differentiation-promoting effect on epidermal cells;

b) Synergetic therapeutic effect of the calcipotriol and nicotinamide, which intensifies the potential therapeutic effect of the composition for various hyperproliferative disorders, including psoriasis;

c) Anti-oxidative effect;

d) Non-toxic formulation approved for topical indications;

e) Convenient “non-messy” formulation which stays in place after application;

f) Calcipotriol in the present formulation does not penetrate the skin, thereby precluding systemic exposure to the drug and concomitant side effects;

g) The calcipotriol-nicotinamide formulation of the invention was classified as a negligible irritant, whereas the commercially available products are labeled as irritants;

h) Avoidance of steroid, corticosteroid and tar-based compounds and concomitant side effects.

### DEFINITIONS

As used herein, the singular forms “a”, “an” and “the” include plural forms unless the context clearly dictates otherwise. Thus, for example, reference to “a PEG” includes combinations of PEG compounds, and so on.

As used herein and in the claims, the term “about” refers to the amount specified plus or minus 10%.

As used herein, a “subject in need thereof” refers to a subject who exhibits at least one clinical sign or symptom of a hyperproliferative scalp disorder, such as psoriasis. Symptoms of scalp psoriasis include one or more of dermatological inflammation; red or white flaky skin; rash; itching; plaques; elevated plaques; hair loss, and blisters.

As used herein, a “therapeutically effective amount” refers to that amount or dosage of the liquid or gel composition according to the invention which is sufficient to substantially inhibit, slow, or reverse the progression of a scalp disease or disorder, thus substantially ameliorating clinical symptoms or substantially preventing the appearance of symptoms associated with a scalp disease or disorder.

As used herein, a “dermatologically acceptable carrier” refers to a carrier and/or a diluent or combination thereof in a pharmaceutical preparation or formulation, that does not cause significant irritation to the skin and does not abrogate the biological activity and properties of the active agent(s) with which it is combined.

As used herein, “formulation” refers to a pharmaceutical preparation that includes a carrier and at least one pharmaceutically active agent. In general, the carrier is selected to produce the desired type of formulation, for example, a gel, a lotion, or a spray. The formulations disclosed herein are generally non-solid dosage forms for topical administration, such as liquids, gels and aerosols. Formulations may be further categorized to further describe the physical state of the components, for example, a hydrogel or an emulsion, and accordingly a single formulation may fit into more than one category or description, for example, a gel formulation may be formulated as a spray.

In the context of the present invention, preferred carriers are those incorporating members of the polyethylene glycol (PEG) family of polymers, a solvent, in particular propylene glycol, and a surfactant.

As used herein, the term “gel” refers to a semisolid consisting of large particles interpenetrated by a liquid (see for example, Physical Pharmacy: Physical Chemical Principles In The Pharmaceutical Sciences, A. Martin, J. Swarbrick, A. Cammarata, Eds., Lea & Febiger, Philadelphia, 4th Edition, 1993). In the present invention, the gel compositions are preferably single-phase gels, meaning that no discernable boundary exists between the polymers and the liquid, since the polymers or other large particles are substantially uniformly distributed throughout the liquid. The polymer particles may be referred to as the dispersed phase, while the liquid may be referred to as the continuous phase. Gels include, without limitation, anhydrous gels, hydrogels, macrogels, microgels, and nanogels, depending for example, on the size of the particles and the presence of water in the gels.

The terms “polyethylene glycol” and “PEG” are used interchangeably herein to refer to polymers of ethylene oxide, having an esterifiable hydroxy group at least at one end of the polymer molecule, as well as derivatives of such polymers having esterifiable carboxy groups.

Polyethylene glycols suitable for the present invention include those having a free hydroxy group at each end of the polymer molecule, those having one hydroxy group etherified with a lower alkyl, e.g., a methyl group, and derivatives of polyethylene glycols having esterifiable carboxy groups. Polyethylene glycols are commercially available under the trade name PEG, usually as mixtures of polymers characterized by an average molecular weight. Polyethylene glycols having an average molecular weight from about 300 to about 5000 are preferred. In a currently preferred embodiment, polyethylene glycols having an average molecular weight of about 400 and of about 4000 are used in the formulations of the invention.

The carrier of the present invention may further include, for example, a thickener, an emollient, an emulsifier, a humectant, a suspending agent, a film forming agent, a foam building agent, a preservative, an antifoaming agent, a fragrance, a lower monoalcoholic polyol, a high boiling point solvent, a propellant, a colorant, a pigment or mixtures thereof.

It is to be explicitly understood that percentages recited herein with respect to a particular component relate to the percentage of that component in relation to the total composition.

### Formulations

The liquid and gel pharmaceutical compositions of the present invention comprise:

calcipotriol at a concentration of about 10 μg/g (micrograms per gram) to about 100 μg/g;

nicotinamide at a concentration of about 10 mg/g (milligrams per gram to about 25 mg/g; and

a dermatologically acceptable carrier.

In one embodiment, calcipotriol is provided at a concentration of about 20 μg/g to about 80 μg/g. In another embodiment, calcipotriol is provided at a concentration of about 30 mg/g to about 60 μg/g. In a particular embodiment, calcipotriol is provided at a concentration of about 50 μg/g.

In a preferred embodiment, nicotinamide is provided at a concentration of about 10 mg/g to about 20 mg/g. In a more preferred embodiment, nicotinamide is provided at a concentration of about 10 mg/g to about 18 mg/g. In other preferred embodiments, nicotinamide is provided at a concentration of about 12 mg/g to about 16 mg/g, or about 13 mg/g to about 15 mg/g. In a currently preferred embodiment, nicotinamide is provided at a concentration of about 14 mg/g.

In a currently preferred embodiment, calcipotriol is provided at a concentration of about 50 μg/g, and nicotinamide is provided at a concentration of about 14 mg/g.

According to the invention, the carrier comprises polyethylene glycol (PEG)-400 at a concentration of about 75% to about 95% (w/w), an additional solvent at a concentration of about 2% to about 25% (w/w), a surfactant at a concentration of about 1% to about 5% (w/w), and optionally, PEG-4000 at a concentration of about 0.1% to about 12% (w/w).

In general, the liquid compositions may be substantially devoid of PEG-4000 or alternatively, comprise PEG-4000 at a relatively low concentration, for example, about 0.1% to about 0.5%. In general, the gel compositions comprise PEG-4000 at concentration of about 1% to about 12%, for example, about 1%, or about 2%, or about 3%, or about 4%, or about 5%, or about 6%, or about 7%, or about 8%, or about 9%, or about 10%, or about 11%, or about 12%. In one embodiment, PEG-400 is provided at a concentration of about 78% to about 93% (w/w). In particular embodiments, PEG-400 is provided at a concentration of about 88%, or about 90%, or about 93%.

Suitable solvents include, but are not limited to propylene glycol, polypropylene glycol, glycerin, sorbitol esters, 1,2,6-hexanetriol, ethanol, isopropanol, diethyl tartrate, butanediol, water and mixtures thereof. In a currently preferred embodiment, the solvent is propylene glycol. In one embodiment, propylene glycol is provided at a concentration of about 2% to about 25% (w/w). In particular embodiments, propylene glycol is provided at a concentration of about 5%, or about 10%, or about 20%.

Suitable surfactants include for example, polyethoxylated alcohols e.g. Steareth-21; fatty alcohols e.g. cetyl/stearyl alcohol; polyoxyethylene glycol esters; polyoxethylene sorbitan fatty acid esters e.g. polysorbate-60, and hydrogenated castor oil; cetyl trimethyl ammonium bromide; cetyl trimethyl ammonium chloride; alkyl benzene sulphonates, sodium dodecyl sulfate; sodium sulfosuccinate; sodium lauryl sulfate; an alkyl naphthalene sulfonate condensate sodium salt; sodium stearate; egg lecithin; soya bean lecithin; synthetic saturated lecithins e g dimyristoyl phosphatidyl choline; synthetic unsaturated lecithins e.g. dioleyl phosphatidyl choline; ethoxylated sorbitan esters; sorbitan esters; polyglycerol esters; sucrose esters; poloxamers; alkyl polyglucosides; polyalkyleneoxide modified heptamethyltrisiloxanes; allyloxypolyethylene glycol methylethers and mixtures thereof. In a particular embodiment, the surfactant is polyoxyethylated stearyl alcohol (Steareth-20). In one embodiment, the Steareth-20 is provided at a concentration of about 2% (w/w).

The composition of the invention may be in the form of a gel, for example, a hydrogel, a macrogel, a microgel, or a nanogel. The gel may further be in the form of an emulsion, a microemulsion, a suspension, or a spray. It is to be explicitly understood that the gel formulations may be classified in more than one of the aforementioned categories.

In one exemplary gel composition of the invention, the carrier comprises PEG-400 at a concentration of about 78% to about 93% (w/w), PEG-4000 at a concentration of about 1% to about 12% (w/w) and propylene glycol at a concentration of about 5% (w/w).

The composition of the invention may be in the form of a liquid, for example, an aqueous solution, a non-aqueous solution, a lotion, or a spray. The liquid may further be in the form of an emulsion or a microemulsion. It is to be explicitly understood that the liquid formulations may be classified in more than one of the aforementioned categories.

In one exemplary liquid composition of the invention, the carrier comprises PEG-400 at a concentration of about 78% to about 93% (w/w), PEG-4000 at a concentration of about 0.1% to about 0.5% (w/w) and propylene glycol at a concentration of about 5% (w/w). In another embodiment, the carrier of the liquid composition comprises PEG-400 at a concentration of about 78% to about 93% (w/w), propylene glycol at a concentration of about 5% (w/w) to about 20% (w/w) and is substantially devoid of PEG-4000.

In particular embodiments, the liquid and gel compositions of the present invention consists essentially of calcipotriol, nicotinamide, PEG-400, PEG-4000, propylene glycol, Steareth-20 and vitamin E.

In certain embodiments, the gel composition has the formulation:
about 10 μg/g to about 100 μg/g calcipotriol;
about 10 to about 18 mg/g nicotinamide;
about 75% to about 95% (w/w) PEG-400;
about 1% to about 12% (w/w) PEG-4000;
about 2% to about 25% (w/w) propylene glycol;
about 1% to about 5% (w/w) Steareth-20; and
about 0.1 to about 1% (w/w) vitamin E.

In one embodiment, the gel composition has the formulation:
about 50 μg/g calcipotriol;
about 14 mg/g nicotinamide;
about 75% to about 95% (w/w) PEG-400;
about 1% to about 12% (w/w) PEG-4000;
about 2% to about 25% (w/w) propylene glycol;
about 1% to about 5% (w/w) Steareth-20; and
about 0.1 to about 1% (w/w) vitamin E.

In a specific embodiment, the gel composition has the formulation:
calcipotriol at a concentration of about 50 μg/g;
nicotinamide at a concentration of about 14 mg/g;
PEG-400 at a concentration of about 88.7%;
PEG-4000 at a concentration of about 4.0%;
propylene glycol at a concentration of about 5%;
Steareth-20 at a concentration of about 2%; and
vitamin Eat a concentration of about 0.3%.

In another specific embodiment, the gel composition has the following formulation:
calcipotriol at a concentration of about 50 μg/g;
nicotinamide at a concentration of about 14 mg/g;
PEG-400 at a concentration of about 89.7%;
PEG-4000 at a concentration of about 3.0%;
propylene glycol at a concentration of about 5%;
Steareth-20 at a concentration of about 2%; and
vitamin E at a concentration of about 0.3%.

In another specific embodiment, the gel composition has the formulation:
calcipotriol at a concentration of about 50 μg/g;
nicotinamide at a concentration of about 14 mg/g;
PEG-400 at a concentration of about 91.7%;
PEG-4000 at a concentration of about 1.0%;
propylene glycol at a concentration of about 5%;
Steareth-20 at a concentration of about 2%; and
vitamin E at a concentration of about 0.3%.

In one embodiment, the liquid composition has the formulation:
about 50 μg/g calcipotriol;
about 14 mg/g nicotinamide;
about 75% to about 95% (w/w) PEG-400;
about 2% to about 25% (w/w) propylene glycol;
about 1% to about 5% (w/w) Steareth-20; and
about 0.1 to about 1% (w/w) vitamin E; and optionally
about 0.1% to about 0.5% (w/w) PEG-4000.

In a particular embodiment, the liquid composition has the formulation:
calcipotriol at a concentration of about 50 μg/g;
nicotinamide at a concentration of about 14 mg/g;
PEG-400 at a concentration of about 92.7%;
propylene glycol at a concentration of about 5%;
Steareth-20 at a concentration of about 2%; and
vitamin E at a concentration of about 0.3%.

In a particular embodiment, the liquid composition has the formulation:
calcipotriol at a concentration of about 50 μg/g;
nicotinamide at a concentration of about 14 mg/g;
PEG-400 at a concentration of about 87.7%;
propylene glycol at a concentration of about 10%;
Steareth-20 at a concentration of about 2%; and
vitamin E at a concentration of about 0.3%.

In a particular embodiment, the liquid composition has the formulation:
calcipotriol at a concentration of about 50 μg/g;
nicotinamide at a concentration of about 14 mg/g;
PEG-400 at a concentration of about 77.7%;
propylene glycol at a concentration of about 20%;
Steareth-20 at a concentration of about 2%; and
vitamin E at a concentration of about 0.3%.

In a particular embodiment, the liquid composition has the formulation:
calcipotriol at a concentration of about 50 μg/g;
nicotinamide at a concentration of about 14 mg/g;
PEG-400 at a concentration of about 92.5%;
PEG-4000 at a concentration of about 0.2%;
propylene glycol at a concentration of about 5%;
Steareth-20 at a concentration of about 2%; and
vitamin E at a concentration of about 0.3%.

In a particular embodiment, a kit comprises the liquid or gel composition of the invention packaged in a container suitable for dispensing of said composition; and written instructions for use.

The liquid composition may be formulated as an aqueous solution, a non-aqueous solution, a suspension, a lotion, a spray, an emulsion, a microemulsion or a combination thereof. The gel composition may be formulated as an anhydrous gel, a hydrogel, a macrogel, a microgel, a nanogel, an emulsion, a microemulsiona suspension, a spray, or a combination thereof.

The gel may further be termed a gel solution or a gel suspension, to describe the physical state of a drug in the gel. In a gel solution, the drug is fully dissolved in the solvent system. In a gel suspension, the drug is mostly suspended in the solvent system, with a small portion of the drug dissolved therein.

The composition of the invention may further comprise a stabilization agent which maintains a drug in suspension within a gel suspension. Examples of stabilization agents include without limitation, sorbitan esters such as monolaurate, monopalmitate, monostearate, trioleate, tristearate, polysorbate 20, polysorbate 40, polysorbate 60, and polysorbate 80; gums such as acacia, tragacanth, xanthan, guar, and veegum; colloids such as carrageenan, alginates, gelatin, agar and bentonite; copolymers and derivatives of: polyvinylpyrrolidone, polyvinyl alcohol, cellulose, polyethylene glycol, polyoxyethylene, poloxamers, carbomers, chitosan, sodium lauryl sulfate, oleic acid and its salts and esters, N-acetyl-N-ethyl morpholinum ethosulfate, and glycerol monstearate.

When the compositions of the invention are formulated as an emulsion, the proportion of the fatty phase may range from about 5% to about 80% by weight, and preferably from about 5% to about 50% by weight, relative to the total weight of the composition. Oils, emulsifiers and co-emulsifiers incorporated in the composition in emulsion form are selected from among those known to those with skill in the art.

Suspensions may comprise suspending agents or thickeners in addition to the active compounds. Suitable suspending agent or thickeners include but not limited to the group consisting of cellulose derivatives like methylcellulose, hydroxyethylcellulose and hydroxypropyl cellulose, alginic acid and its derivatives, xanthan gum, guar gum, gum arabic, tragacanth, gelatin, acacia, bentonite, starch, microcrystalline cellulose, povidone and mixtures thereof.

Aqueous suspensions may optionally contain additional excipients selected from the group consisting of wetting agents, flocculating agents, thickeners, and the like.

Suitable wetting agents include but not limited to the group consisting of glycerol polyethylene glycol, polypropylene glycol and mixtures thereof, and surfactants. The concentration of the wetting agents in the suspension should be selected to achieve optimum dispersion of the pharmaceutical powders within the suspension with the lowest feasible concentration of the wetting agent.

Suitable flocculating agents are exemplified by but not limited to the group consisting of electrolytes, surfactants, and polymers.

The suspending agents, wetting agents and flocculating agents are provided in amounts that are effective to form a stable suspension of the pharmaceutically effective agent.

The composition of the invention may be provided as a spray formulation. As used herein, the term “spray” refers to a jet or mass of finely divided liquid particles or droplets. Spray formulations include propellant spray aerosols and mechanical pump spray aerosols.

Propellant spray aerosols comprise a suitable propellant i.e. a substance that is a gas under atmospheric conditions but a liquid when under pressure. Examples of pharmaceutically acceptable propellants include dimethyl ether, diethyl ether, fluorocarbons such as 1,1-difluoroethane, hydrocarbons such as butane or propane, liquified gases such as nitrogen or carbon dioxide and mixtures thereof. A propellant spray aerosol is a formulation consisting of both a gel preparation and a pharmaceutically acceptable propellant under pressure.

A mechanical pump spray aerosol is a formulation that does not contain a propellant and is ejected from a closed container by means of mechanical force i.e. by pushing down a finger-activated accuator.

A spray aerosol is conveniently provided in a spray dispenser, so that the formulation may be delivered to the scalp in the form of a fine mist which settles as a gel. As used herein, a “spray dispenser” refers to a container which holds the formulation of the invention, and includes a mechanism for ejecting the formulation in the form of a spray. The mechanism is typically activated by the application of finger pressure onto an accuator or nozzle head.

The hand held spray dispenser of the device may be any type of spray dispenser known in the art, which typically includes a nozzle having an outlet aperture through which the formulation is expelled or ejected in the form of a spray upon depression of the nozzle, for example, by application of finger pressure. The activation of the device by depression of the nozzle may also be referred to as “actuation”.

A pump-type spray dispenser dispenses the formulation under normal atmospheric pressure; application of finger pressure temporarily pressurizes the formulation to cause a portion of it to leave the dispenser as a spray. The pressure in the mechanism returns to atmospheric soon after the portion of formulation has been dispensed. Pump-type spray dispensers are disclosed, for example in U.S. Pat. Nos. 3,159,316; 4,034,900, and 4,050,860. A pump-type spray dispenser for administering medicaments to the ear is disclosed for example, in U.S. Pat. No. 5,176,654.

A propellant aerosol spray dispenser contains the formulation to be dispensed, and a gas under pressure, typically held within a metal container (made for example, from aluminum or tinplate) which can withstand pressure higher than atmospheric pressure. The formulation is typically a liquid or gel in mono-phasic solution (i.e. homogeneous solution) or in bi-phasic solution (i.e. aqueous solution and oil solution). The container is tightly closed with a valve orifice, and then an aerosol propellant (i.e. a liquefied gas), such as butane, propane, a hydrofluoroalkane mixture or any other propellant as is known in the art, is inserted, thus creating pressure inside the container (e.g. 3 atmospheres). Agitation of the container, even minimally, causes the gas and liquid to mix; depression of the nozzle results in the ejection of the pressurized formulation. Propellant aerosol spray dispensers are disclosed, for example, in U.S. Pat. Nos. 5,322,683; 5,397,564; and 6,730,288. The spray dispenser may be a metered dose spray dispenser. Such a dispenser expels a pre-determined volume of the formulation i.e. a metered dose, with each actuation of the device. Metered dose devices are known in the art for different applications, described for example, in U.S. Pat. Nos. 6,032,836; 5,697,532; 5,502,076, and 6,702,155, and in US Patent Application No. 2003/0178022.

The propellant aerosol spray dispenser may further be a bag-on-valve spray dispenser. A bag-on-valve spray dispenser dispenses the formulation under the force of a propellant which remains separated from the formulation within the dispenser. In a bag-on-valve system, the formulation is contained within a welded bag within a conventional aerosol can. Compressed air or gas in the aerosol can is on the outside of the bag and acts as a propellant on the product inside the bag. Compared to conventional aerosol spray dispensing, in which a product and propellant require mixing for an optimized spray effect, the separation of these two components in the bag-on-valve system offers many advantages, including the use of compressed air or liquefied propellants, suitability for oxygen sensitive products, product emptying up to 99%, use of the aerosol can in all positions, non-chilling product discharge, and reduced need for preservatives since the product in the bag does not contact oxygen.

Lotions may include at least one or more emollient, which can function as either or both a lubricating and thickening agent. The emollients can comprise in total from about 0.1% to about 50%, preferably from about 1% to about 10%, by weight of the composition. Any emollients known to those of skill in the art as suitable for topical application to human skin may be used. These include, but are not limited to: hydrocarbon oils and waxes, including mineral oil, petrolatum, paraffin, ceresin, ozokerite, microcrystalline wax, polyethylene, and perhydrosqualene; silicone oils; triglyceride fats and oils, including those derived from vegetable, animal and marine source; including jojoba oil and shea butter; acetoglyceride esters, such as acetylated monoglycerides; ethoxylated glycerides, such as ethoxylated glyceryl monostearate; fatty acids, fatty alcohols and derivatives thereof. Other suitable emollients include lanolin and lanolin derivatives; polyhydric alcohols and polyether derivatives; polyhydric alcohol esters; wax esters; vegetable waxes; phospholipids, such as lecithin and derivatives; sterols, including, but not limited to, cholesterol and cholesterol fatty acid esters; amides, such as fatty acid amides, ethoxylated fatty acid amides, and solid fatty acid alkanolamides.

The lotions may further contain from about 1% to about 5%, more preferably from 2% to 5%, of an emulsifier (also referred to herein as a surfactant). The emulsifiers can be nonionic, anionic or cationic. The choice of suitable emulsifiers may be readily determined by those of skill in the art.

Other conventional components of lotions may be included. One such additive is a thickening agent in an amount of about 1% to 10% of the composition. Examples of suitable thickening agents include, but are not limited to: cross-linked carboxypolymethylene polymers, ethyl cellulose, polyethylene glycols, gum tragacanth, gum karaya, xanthan gums, bentonite and other clays, hydroxyethyl cellulose, and hydroxypropyl cellulose.

The lotions are formulated by simply admixing all of the components together. The active ingredients are dissolved, suspended or otherwise uniformly dispersed in the mixture.

The composition of the invention may additionally include an anti-fungal agent, which is especially useful for the treatment of hyperproliferative skin diseases of the scalp which are complicated by fungal infections. Non-limiting examples of an anti-fungal agents include miconazol, clotrimazol, terbinafin, ciclopirox, bifonazol, nystatin, ketoconazol, econazol, and amorolfine.

The present formulations may also include additives, as are known in the art, such as opacifiers, antioxidants, fragrance, colorant, gelling agents, thickening agents, stabilizers, surfactants, preservatives (antimicrobial agents) and the like. In particular, an antioxidant may be added to the composition in a concentration range of about 0.1% to about 10% (w/w). Suitable antioxidants include ascorbic acid (vitamin C) and its salts, and tocopherol (vitamin E). In one embodiment, the composition comprises vitamin E at a concentration of about 0.1% to about 1% (w/w), for example 0.3% (w/w).

Preservatives may be included within the compositions to prevent spoilage caused by growth of microbes such as bacteria, yeasts and molds. Suitable preservatives are typically selected from methyl and propyl esters of p-hydroxybenzoic acid (i.e., methyl and propyl paraben), sodium benzoate, sorbic acid, imidurea, and combinations thereof.

It is to be noted that unless otherwise specified, percentages stated herein are in relation to the final weight of the composition. It is further to be noted that all numerical values stated herein include a standard deviation that is acceptable in the pharmaceutical arts, and appropriate for any particular component or pharmaceutical ingredient.

The description herein of concentrations, amounts, etc., of various components of the invention in a range format is merely for convenience and brevity, and should not be construed as an inflexible limitation on the scope of the invention. Accordingly, the description of a range should be considered to have specifically disclosed all the possible subranges as well as individual numerical values within that range. For example, description of a range such as 6% to 12% should be considered to have specifically disclosed subranges such as 6% to 7%, 7% to 8%, 7% to 9%, 6% to 9%, 9% to 12%, 9% to 11% etc., as well as individual numbers within that range, for example, 8%, 10%, 11% etc. This applies regardless of the breadth of the range and in all contexts throughout this application.

### Methods of Treatment and Uses of the Compositions

The present invention further provides a method of preventing or treating a hyperproliferative skin disease or disorder of the scalp. Scalp diseases and disorders which may be treated by the method of the invention include psoriasis, solar (actinic) keratosis, naevus sebaceous, seborrhoeic keratoses, atopic dermatitis and lichen planus.

The method of the invention comprises topically administering to the scalp of a subject in need thereof a therapeutically effective amount of a gel composition, the composition comprising calcipotriol at a concentration of about 10 μg/g to about 100 μg/g; nicotinamide at a concentration of about 10 mg/g to about 25 mg/g; and a dermatologically acceptable carrier, wherein the carrier comprises PEG-400 at a concentration of about 75% to about 95% (w/w); propylene glycol at a concentration of about 2% to about 25% (w/w), a surfactant at a concentration of about 1% to about 5% (w/w), and optionally, PEG-4000 at a concentration of about 0.1% to about 12% (w/w).

In some of the gel compositions, the carrier comprises PEG-400 at a concentration of about 78% to about 93% (w/w), PEG-4000 at a concentration of about 1% to about 12% (w/w), and propylene glycol at a concentration of about 5% (w/w).

In some of the liquid compositions, the carrier comprises PEG-400 at a concentration of about 78% to about 93% (w/w), PEG-4000 at a concentration of about 0.1% to about 0.5% (w/w) and propylene glycol at a concentration of about 5% (w/w). In some of the liquid compositions, the carrier comprises PEG-400 at a concentration of about 78% to about 93% (w/w), propylene glycol at a concentration of about 5% (w/w) to about 20% (w/w) and the composition is substantially devoid of PEG-4000.

Other particular embodiments of the liquid and gel compositions are as hereinbefore described.

As described herein, the gel composition may be provided in the form of a hydrogel, a macrogel, a microgel, a nanogel, an emulsion, a microemulsiona suspension, a spray, or a combination thereof. The liquid composition may be in the form of an aqueous solution, a non-aqueous solution, a suspension, a lotion, a spray, an emulsion, a microemulsion or a combination thereof. The topical administration to the scalp may be carried out up to about four times per day. In a particular embodiment, the administration is carried out once or twice daily. In another embodiment, the administration is carried out twice daily at intervals of about 12 hours. In another embodiment, the administration is carried out once daily. In another embodiment, the administration is carried out three to five times per week.

The method may be carried out for a period of about 4 weeks to about 52 weeks, for example, about 6 weeks to about 12 weeks, or about 12 weeks to about 26 weeks.

In a particular embodiment, the subject is a mammal. In a particular embodiment, the subject is a human.

In another aspect, the invention provides the use of calcipotriol at a concentration of about 10 μg/g to about 100 μg/g; nicotinamide at a concentration of about 10 mg/g to about 25 mg/g; and a carrier, wherein the carrier comprises PEG-400 at a concentration of about 75% to about 95% (w/w); PEG-4000 at a concentration of about 1% to about 12% (w/w); propylene glycol at a concentration of about 2% to about 25% (w/w); a surfactant at a concentration of about 1% to about 5% (w/w); and optionally, PEG-4000 at a concentration of about 0.1% to about 12% (w/w) for the preparation of a liquid composition for the topical treatment of a hyperproliferative skin disease or disorder of the scalp.

The following examples are to be considered merely as illustrative and non-limiting in nature. It will be apparent to one skilled in the art to which the present invention pertains that many modifications, permutations, and variations may be made without departing from the scope of the invention.

### EXAMPLES

### Example 1

### Gel Formulations

Four gel formulations according to the invention are presented in Table 1.

TABLE 1

Formulation

| Component | I | II | III | IV |
|---|---|---|---|---|
| Calcipotriol (μg/g) | 50 | 50 | 50 | 50 |
| Nicotinamide (mg/g) | 14 | 14 | 14 | 14 |
| PEG-400 (% w/w) | 91.7 | 90.7 | 89.7 | 88.7 |
| PEG-4000 (% w/w) | 1 | 2 | 3 | 4 |
| Propylene glycol (% w/w) | 5 | 5 | 5 | 5 |
| Steareth-20 (% w/w) | 2 | 2 | 2 | 2 |
| Vitamin E (% w/w) | 0.3 | 0.3 | 0.3 | 0.3 |

### Example 2

### Liquid Formulations

Four liquid formulations according to the invention are presented in Table 2.

TABLE 2

Formulation

| Component | A | B | C | D |
|---|---|---|---|---|
| Calcipotriol (μg/g) | 50 | 50 | 50 | 50 |
| Nicotinamide (mg/g) | 14 | 14 | 14 | 14 |
| PEG-400 (% w/w) | 92.5 | 92.7 | 87.7 | 77.7 |
| PEG-4000 (% w/w) | 0.2 | — | — | — |
| Propylene glycol (% w/w) | 5 | 5 | 10 | 20 |
| Steareth-20 (% w/w) | 2 | 2 | 2 | 2 |
| Vitamin E (% w/w) | 0.3 | 0.3 | 0.3 | 0.3 |

### Example 3

### Stability Studies

The formulations prepared in Example 1 and Example 2 are subjected to an accelerated stability test at 40° C. Samples are taken at 2 weeks, 1 month, 2 months, 3 months, 6 months and 12 months and analyzed.

### Example 4

### Human Trials

Patients having scalp psoriasis are divided into several trial arms and patients are treated by topical application to the scalp with predetermined compositions twice daily, morning and night. The predetermined compositions used for treating the trial arms include the gel and liquid formulations prepared according to Example 1 and Example 2, respectively. In addition, the trial arms include a placebo arm and other combinations of nicotinamide and calcipotriol known in the art. The patients are instructed to report all adverse events, whether mild and severe, which occur during treatment. Treatment efficacy is determined by evaluating psoriatic symptoms at the end of the study period. Psoriatic symptoms (i.e. scaling, erythema and plaque formation) are determined and patients are considered as having a positive outcome if they are classified as clear or almost clear of disease symptoms. Dermal irritation, including production of inflammatory changes or any damage in the skin, is determined. In addition, dermal corrosion is monitored, including visible necrosis, ulcers and bleeding.

Assessment of patients' outcome following treatment includes: PGA (Physician's Global Assessment) score follow-up—a seven-point scale from clear to severe is used, which is depicted in Table 1;

TABLE 1
PGA Scoring

| | |
|---|---|
| Score 7 | Severe: Very marked plaque elevation, scaling and/or erythema. |
| Score 6 | Moderate to Severe: Marked plaque elevation, scaling and/or |
| | erythema. |
| Score 5 | Moderate: Moderate plaque elevation, scaling and/or erythema. |
| Score 4 | Mild to moderate: Intermediate between moderate and mild. |
| Score 3 | Mild: Slight plaque elevation, scaling and/or erythema. |
| Score 2 | Almost clear: Intermediate between mild and clear. |
| Score 1 | Clear: No sign of psoriasis. |

Symptom score: Erythema (E), Induration (I) and Scaling (S) is scored using the scale shown in table 2, hereinbelow.

TABLE 2
Symptom scores

| Score | Definition |
|---|---|
| 0 | None |
| 1 | Mild |
| 2 | Moderate |
| 3 | Severe |
| 4 | Very severe |

Patient Self-Assessment: The patient is required to generally assess tolerance and treatment effect by general scoring (of the efficacy and tolerance). The Patient's Self Assessment has 5 graded definitions (used both for (i) efficacy (Table 3) and (ii) tolerance (Table 4)):

TABLE 3
Efficacy score

| Efficacy Score | Definition |
|---|---|
| 1 | VERY POOR |
| 2 | POOR |
| 3 | DON'T KNOW |
| 4 | GOOD |
| 5 | VERY GOOD |

TABLE 4
Tolerance score

| Tolerance Score | Definition |
|---|---|
| 1 | VERY POOR |
| 2 | POOR |
| 3 | DON'T KNOW |
| 4 | GOOD |
| 5 | VERY GOOD |

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1.-52. (canceled)

53. A pharmaceutical composition for topical administration to the scalp, the composition comprising:
calcipotriol at a concentration of about 10 μg/g to about 100 μg/g;
nicotinamide at a concentration of about 10 mg/g to about 25 mg/g; and
a dermatologically acceptable carrier, wherein the carrier comprises:
polyethylene glycol (PEG)-400 at a concentration of about 75% to about 95% (w/w), an additional solvent at a concentration of about 2% to about 25% (w/w), a surfactant at a concentration of about 1% to about 5% (w/w), and optionally, polyethylene glycol (PEG)-4000 at a concentration of about 0.1% to about 12% (w/w).

54. The composition according to claim 53, wherein the additional solvent is selected from the group consisting of: propylene glycol, polypropylene glycol, glycerin, sorbitol esters, 1,2,6-hexanetriol, ethanol, isopropanol, diethyl tartrate, butanediol, water and mixtures thereof.

55. The composition according to claim 54, wherein the solvent is propylene glycol.

56. The composition according to claim 53, wherein the surfactant is selected from the group consisting of: a polyethoxylated alcohol; a fatty alcohol e.g. cetyl/stearyl alcohol; a polyoxyethylene glycol ester; a polyoxethylene sorbitan fatty acid ester e.g. polysorbate-60; hydrogenated castor oil; cetyl trimethyl ammonium bromide; cetyl trimethyl ammonium chloride; an alkyl benzene sulphonate, sodium dodecyl sulfate; sodium sulfosuccinate; sodium lauryl sulfate; an alkyl naphthalene sulfonate condensate sodium salt; sodium stearate; egg lecithin; soya bean lecithin; a synthetic saturated lecithin e.g. dimyristoyl phosphatidyl choline; a synthetic unsaturated lecithin e.g. dioleyl phosphatidyl choline; an ethoxylated sorbitan ester; a sorbitan ester; a polyglycerol ester; a sucrose ester; a poloxamer; an alkyl polyglucoside; a polyalkyleneoxide modified heptamethyltrisiloxane; an allyloxypolyethylene glycol methylether and mixtures thereof.

57. The composition according to claim 56, wherein the surfactant is a polyethoxylated alcohol.

58. The composition according to claim 53, in a form selected from the group consisting of a gel and a liquid.

59. The composition according to claim 53, wherein the PEG-400 is provided at a concentration of about 78% to about 93% (w/w).

60. The composition according to claim 53, comprising PEG-4000 at a concentration of about 1% to about 12% (w/w), and wherein the composition is in the form of a gel.

61. The composition according to claim 53, comprising PEG-4000 at a concentration of about 0.1% to about 0.5% (w/w), and wherein the composition is in the form of a liquid.

62. The composition according to claim 53, wherein the composition is substantially devoid of PEG-4000, and wherein the composition is in the form of a liquid.

63. The composition according to claim 58, wherein the composition is in the form of a gel selected from the group consisting of a hydrogel, a macrogel, a microgel, a nanogel, an emulsion, a microemulsion, a suspension, a spray, and combinations thereof.

64. The composition according to claim 58, wherein the composition is in the form of a liquid is selected from the group consisting of an aqueous solution, a non-aqueous solution, a lotion, a spray, an emulsion, and a microemulsion.

65. The composition according to claim 55, comprising PEG-400 at a concentration of about 78% to about 93% (w/w), PEG-4000 at a concentration of about 1% to about 12% (w/w) and propylene glycol at a concentration of about 5% (w/w), wherein the composition is in the form of a gel.

66. The composition according to claim 55, comprising PEG-400 at a concentration of about 78% to about 93% (w/w), PEG-4000 at a concentration of about 0.1% to about 0.5% (w/w) and propylene glycol at a concentration of about 5% (w/w), wherein the composition is in the form of a liquid.

67. The composition according to claim 55, comprising PEG-400 at a concentration of about 78% to about 93% (w/w), propylene glycol at a concentration of about 5% (w/w) to about 20% (w/w), wherein the composition is substantially devoid of PEG-4000, and wherein the composition is in the form of a liquid.

68. The composition according to claim 53, wherein the composition has the formulation:
calcipotriol at a concentration of about 50 μg/g;
nicotinamide at a concentration of about 14 mg/g;
PEG-400 at a concentration of about 88.7%;
PEG-4000 at a concentration of about 4.0%;
propylene glycol at a concentration of about 5%;
Steareth-20 at a concentration of about 2%; and
vitamin E at a concentration of about 0.3%.

69. The composition according to claim 53, wherein the composition has the formulation:
calcipotriol at a concentration of about 50 μg/g;
nicotinamide at a concentration of about 14 mg/g;
PEG-400 at a concentration of about 92.7%;
propylene glycol at a concentration of about 5%;
Steareth-20 at a concentration of about 2%; and
vitamin E at a concentration of about 0.3%.

70. A kit comprising the composition according to claim 53, wherein the composition is packaged in a container suitable for dispensing; and written instructions for use.

71. A method of preventing or treating a hyperproliferative skin disease or disorder of the scalp comprising the step of:
topically administering to the scalp of a subject in need thereof a therapeutically effective amount of a composition comprising calcipotriol at a concentration of about 10 μg/g to about 100 μg/g; nicotinamide at a concentration of about 10 mg/g to about 25 mg/g; and a dermatologically acceptable carrier, wherein the carrier comprises PEG-400 at a concentration of about 75% to about 95% (w/w); propylene glycol at a concentration of about 2% to about 25% (w/w), a surfactant at a concentration of about 1% to about 5% (w/w); and optionally, PEG-4000 at a concentration of about 0.1% to about 12% (w/w);
thereby preventing or treating the hyperproliferative skin disease or disorder of the scalp.

72. The method according to claim 71, wherein the disease or disorder of the scalp is selected from the group consisting of: psoriasis, solar (actinic) keratosis, naevus sebaceous, seborrhoeic keratoses, atopic dermatitis and lichen planus.
